(19) United States
(12) Patent Application Publication
(10) Pub. No.: US 2005/0152913 A1
(43) Pub. Date: Jul. 14, 2005

(54) Process for preapring maytansinol

(76) Inventor: Ann Marie Eldridge, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/513,682

(22) PCT Filed: May 12, 2003
(86) PCT No.: PCT/US03/14759

Related U.S. Application Data
(60) Provisional application No. 60/379,976, filed on May 13, 2002.

(51) Int. Cl. A61K 39/395; C07K 16/46

(52) U.S. Cl. 424/178.1; 530/391.1; 540/462

(57) ABSTRACT

Processes for preparing maytansinol from disulfide-containing maytansinoid esters by reduction with LiAlH₄. The maytansinol is useful for preparing cell-binding agent/maytansinoid complexes.

### FIELD OF THE INVENTION

The present invention relates to a process for preparing maytansinol from disulfide-containing maytansinoid esters.

### BACKGROUND OF THE INVENTION

Highly cytotoxic maytansinoid drugs and their therapeutic use have been described in U.S. Pat. No. 5,208,020. These drugs link cytotoxic maytansinoids to cell-binding agents such as antibodies and are useful as tumor-activated pro-drugs.

These immunoconjugate drugs can be prepared from maytansinoid esters coupled to antibodies through a disulfide linkage. The maytansinoid esters are prepared from maytansinol and its ansamitocin precursors as described in U.S. Pat. No. 6,333,410. Ansamitocin production from Actinosynnema fermentation is described in PCT Publication No. WO01/77360 and U.S. Pat. Nos. 4,162,940; 4,228,239; 4,356,265; and 4,450,234.

Maytansinol is an expensive starting material and its precursor sources are limited to fermentation processes. The reaction conditions used to produce maytansinoid esters produce roughly equal amounts of the desired stereoisomer, an undesired stereoisomer and unreacted maytansinol. Thus, it would be desirable to recover maytansinol from the undesired stereoisomer produced in the reaction for use in subsequent esterification reactions.

### SUMMARY OF THE INVENTION

One aspect of the invention is processes for the preparation of maytansinol comprising reducing disulfide-containing maytansinoid esters with lithium aluminum hydride.

Another aspect of the invention is cell-binding agent/maytansinoid complexes prepared from maytansinol produced by the processes of the invention.

### DETAILED DESCRIPTION OF THE INVENTION

All publications, including but not limited to patents and patent applications, cited in this specification are herein incorporated by reference as though fully set forth.

The present invention provides a process for preparing maytansinol by reduction of the maytansinoid ester D-DM1-SMe (IV) with lithium aluminum hydride (LiAlH₄). D-DM1-SMe is an undesired isomer produced in the esterification of maytansinol by amino acid derivatives. The recovered maytansinol can be recycled to produce more of the desired L-DM1-SMe isomer (III).

The present invention also provides antibody/maytansinoid complexes prepared from maytansinol produced by the process of the invention.

Disulfide-containing maytansinoid esters useful for preparing the thiol-containing maytansinoid ester DM1 can be prepared by reacting the N-methyl-L-alanine derivative N-methyl-N-(3-methyldithiopropanoyl)-L-alanine (II) with maytansinol (I) in the presence of dicyclohexylcarbodiimide (DCC) and zinc chloride (ZnCl₂) as shown in Scheme 1. The reaction yields approximately equal amounts of the desired isomer L-DM1-SMe (III), an undesired isomer R-DM1-SMe (IV) and unreacted maytansinol. The unreacted maytansinol is separated by chromatography from the mixture of isomers and the desired L-isomer is then separated from the R-isomer by a separate chromatography. L-DM1-SMe is reduced with dithiothreitol (DTT) to produce DM1 (Scheme 2) which is useful for preparing immunoconjugate drugs.

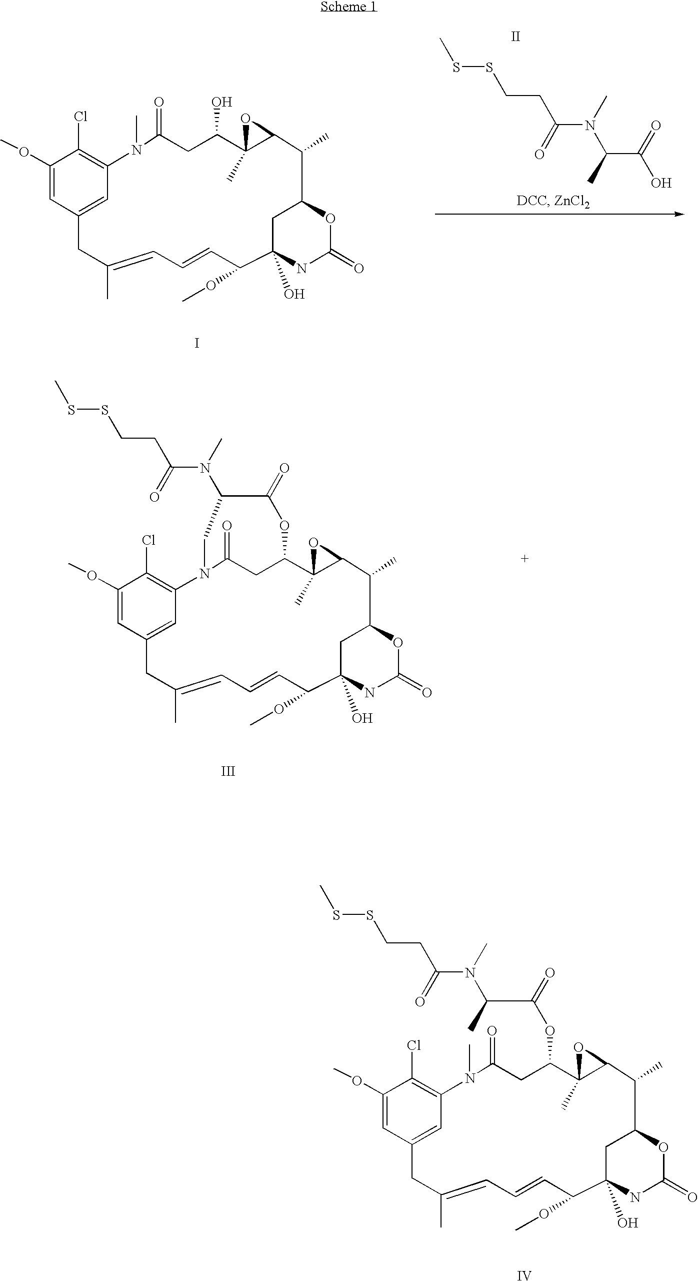

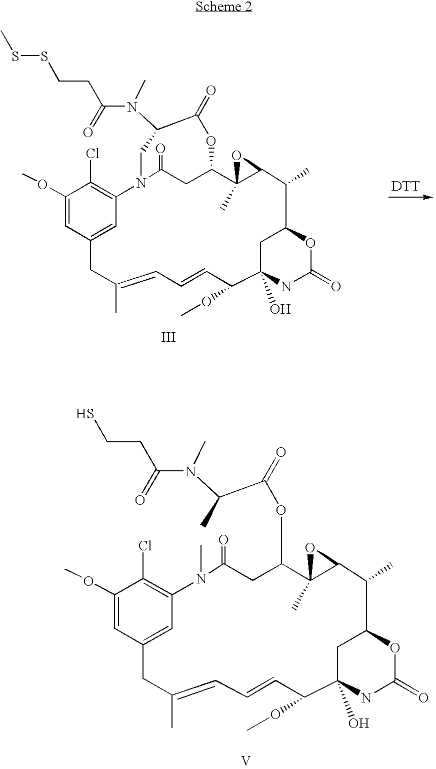

The process of the invention comprises the step of reacting D-DM1-SMe (IV) with LiAlH₄ in a reductive reaction as shown in Scheme 3. The reaction is carried out in a water-miscible polar organic solvent. Preferably, the reaction temperature is about −5° C. to about 10° C. Most preferably, a solution of D-DM1-SMe (IV) in tetrahydrofuran (THF) at about 20° C. is added to a THF solution of LiAlH₄ cooled to about −5° C.

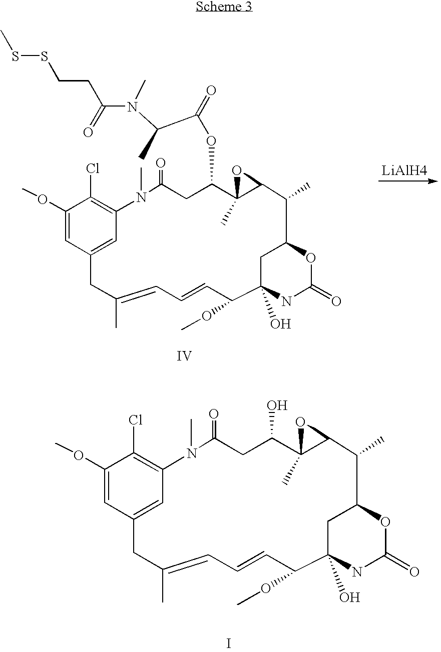

The D-aminoacyl ester of maytansinol, D-DM1-SMe (IV), is prepared from maytansinol by esterification with disulfide-containing N-methyl-L-alanine derivatives such as Compound II, in the presence of DCC and ZnCl₂ as disclosed in U.S. Pat. No. 6,333,410. L- and D-aminoacyl esters of maytansinol containing a disulfide-linking group are produced. The stereoisomers are separated and D-DM1-SMe collected for use in the process of the invention.

LiAlH₄ and THF can be purchased from chemical supply houses such as Aldrich Chemical Co. (St. Louis, Mo.).

The process of the invention can be used to make cell-binding agent/maytansanoid complexes by converting a compound of Formula I prepared by the process of the invention into the cell-binding agent/maytansinoid complex. Compounds of Formula I produced by the process of the invention can be converted into a cell-binding agent/maytansinoid complex as described in U.S. Pat. No. 5,208,020 to produce N-methyl-L-alanine containing maytansinoid derivatives. These derivatives are then conjugated to cell-binding agents, preferably antibodies, via various linkers and are useful as tumor-activated pro-drugs. Preferably, the linkage is a disulfide link.

An exemplary cell-binding agent/maytansinoid complex can be prepared by a process comprising the following steps:
(a) esterifying maytansinol prepared by the process of claim 1 with a compound of Formula II to form a disulfide-containing maytansinoid ester;
(b) reducing the disulfide-containing maytansinoid ester prepared by step (a) to a thiol-containing maytansinoid;
(c) introducing dithiopyridyl groups into a cell-binding agent; and
(d) linking the thiol-containing maytansinoid produced by step (b) to the dithiopyridyl cell-binding agent of step (c) by a disulfide link.

The present invention will now be described with reference to the following specific, non-limiting example.

### EXAMPLE

### Preparation of Maytansinol from D-DM1-SMe

All reagents utilized herein were sourced as first described below.

A 15 mL septum-capped vial, equipped with a magnetic stirrring bar, was charged with lithium aluminum hydride (1M in THF, 0.52 mL, 5.19×10⁻⁴ mole, 4.2 equiv, Aldrich Chemical Co., St. Louis, Mo.) and the solution cooled to −5° C. A solution of N-methyl-N-(3-methyldithiopropanoyl)-D-alanylmaytansine (100 mg, 1.28×10⁻⁴ mole, 1 equiv, ChemSyn Laboratories, Lenexa, Kans.) in anhydrous THF (5 mL, Aldrich Chemical Co.) was added at such a rate that the temperature did not exceed 8° C. The reaction mixture was cooled back to −5° C. and the progress of the reaction was monitored by high performance liquid chromotography (HPLC). After 2 h the reaction was complete, the HPLC assay showing maytansinol as the major product, 95.9% by peak area. The product matched the retention times of maytansinol in two different HPLC systems and had the desired molecular mass by LC/MS. The reaction can be worked up by known standard techniques. See Reagents for Organic Synthesis, Volume 1, Feiser and Feiser, pp 583-584, 1967.

The present invention may be embodied in other specific forms without departing from the spirit or essential attributes thereof, and, accordingly, reference should be made to the appended claims, rather than to the foregoing specification, as indicating the scope of the invention.

1) a process for the preparation of maytansinol comprising reducing D-DM1-SMe with lithium aluminum hydride.

2) The process of claim 1 wherein D-DM1-SMe is added to at least about 4 equivalents of lithium aluminum hydride.

3) The process of claim 1 wherein the lithium aluminium hydride is in tetrahydrofuran (THF).

4) The process of claim 4 wherein the reaction temperature is at about −5° C. to about 10° C.

5) The process of claim 1 wherein about 96% of D-DM1-SMe is converted to maytansinol.

6) Maytansinol prepared by the process of claim 1.

7) A cell-binding agent/maytansinoid complex prepared by converting maytansinol prepared by the process of claim 1 into the cell-binding agent/maytansinoid complex.

8) The cell-binding agent/maytansinoid complex of claim 7 wherein the cell-binding agent is an antibody.

9) A cell-binding agent/maytansinoid complex prepared by a process comprising the following steps:
(a) esterifying-maytansinol prepared by the process of claim 1 with a compound of Formula II to form a disulfide-containing maytansinoid ester;
(b) reducing the disulfide-containing maytansinoid ester prepared by step (a) to a thiol-containing maytansinoid;
(c) introducing dithiopyridyl groups into a cell-binding agent; and
(d) linking the thiol-containing maytansinoid produced by step (b) to the dithiopyridyl cell-binding agent of step (c) by a disulfide link.

10) The cell-binding agent/maytansinoid complex of claim 8 wherein the cell-binding agent is an antibody.
